(19) United States
(12) Patent Application Publication
(10) Pub. No.: US 2009/0099240 A1
(43) Pub. Date: Apr. 16, 2009

(54) METHODS FOR TREATING ENERGY METABOLISM DISORDERS BY INHIBITING FATTY ACID AMIDE HYDROLASE ACTIVITY

(75) Inventors: Jeff A. Parrott, Irvine, CA (US); Timothy R. Compton, Issaquah, WA (US); Olivier Dasse, Foothill Ranch, CA (US); David Putman, Irvine, CA (US)

Correspondence Address: ORGANON USA, INC.;c/o Schering-Plough Corporation, 2000 Galloping Hill Road, Mail Stop: K-6-1, 1990, Kenilworth, NJ, 07033, US

(73) Assignee: N.V. Organon, Oss (NL)

(21) Appl. No.: 11/866,128

(22) Filed: Oct. 2, 2007

Related U.S. Application Data
(60) Provisional application No. 60/827,861, filed on Oct. 2, 2006.

(51) Int. Cl. A61K 31/455; A61K 31/216; A61P 3/00; A61P 3/10; A61P 3/06; A61P 9/10

(52) U.S. Cl. 514/356; 514/529

(57) ABSTRACT

Disclosed herein are methods for treating energy metabolism disorders by administering a composition containing a therapeutically effective amount of a fatty acid amide hydrolase inhibitor. The composition can also be administered to reduce body fat, body weight, or caloric intake.

### BACKGROUND

Energy metabolism disorders such as obesity, arteriosclerosis, and diabetes are among the leading causes of morbidity and mortality in the industrialized world. High body fat, body weight, and excessive caloric intake, commonly associated with modern diets, are key risk factors affecting the incidence and outcome of energy metabolism disorders and related conditions.

### SUMMARY OF THE INVENTION

Described herein are methods and compositions for increasing systemic levels of fatty acid amides by inhibiting fatty acid amide hydrolase (FAAH) (i.e., administering an inhibitor of FAAH). Also described herein are methods and compositions for increasing muscle tissue, decreasing body fat, body weight, and caloric intake. One aspect described herein relates to treating an energy metabolism disorder (EMD) by administering (e.g., orally, bucally, transdermally, intranasally, or rectally) a composition containing a therapeutically effective amount of a FAAH inhibitor to a subject (e.g., a human) suffering from the EMD. As used herein, an EMD refers to any health condition, disease, metabolic syndrome or disorder that is driven by or drives energy storage in or release from fat. An EMD can be, e.g., insulin resistance, diabetes, hyperlipidemia, obesity, liver steatosis, steatohepatitis, non-alcoholic steatohepatitis, arteriosclerosis, or atherosclerosis. A person is considered to have a metabolic syndrome when the person has at least two of the following metabolic risk factors:
Abdominal obesity (excessive fat tissue in and around the abdomen);
Atherogenic dyslipidemia (blood fat disorders—high triglycerides, low HDL cholesterol and high LDL cholesterol—that foster plaque buildups in artery walls);
Elevated blood pressure;
Insulin resistance or glucose intolerance (the body can't properly use insulin or blood sugar);
Prothrombotic state (e.g., high fibrinogen or plasminogen activator inhibitor-1 in the blood); and
Proinflammatory state (e.g., elevated C-reactive protein in the blood).

Where the subject is suffering from hyperlipidemia, arteriosclerosis, insulin resistance, diabetes, hyperlipidemia, obesity, liver steatosis, steatohepatitis, or non-alcoholic steatohepatitis, the subject can also be administered a therapeutically effective amount of a drug for lowering circulating cholesterol levels (e.g., a statin, niacin, fibric acid derivative, or bile acid binding resin).

Another aspect relates to reducing body fat by administering a composition containing a therapeutically effective amount of a FAAH inhibitor. A further aspect relates to decreasing body weight by administering a composition containing a cosmetically effective amount of a FAAH inhibitor. Yet another aspect relates to decreasing caloric intake by administering a composition containing a therapeutically effective amount of a FAAH inhibitor.

Where the method is drawn to reducing body fat, body weight, or caloric intake, the subject can also be provided a calorie-restricted diet or an exercise regimen. The subject can also be administered a composition containing a therapeutically or cosmetically effective amount of a weight loss drug, e.g., an appetite suppressant such as diethylpropion, mazindole phendimetrazine, phentermine, or sibutramine; hyperlipidemia drugs (such as statins and PPAR agonists); obesity drugs; fibric acids; lipid up-take blockers (e.g. Orlistat); MCH-1 (Melanin-concentrating hormone) antagonists; cholesterol lowering bile acid sequestrants; HMG CoA Reductase Inhibitors; absorption blockers (e.g., Ezetimibe); or nesfatin-1 agonists.

In any of the methods described herein, the FAAH inhibitor to be administered to the subject (e.g., a human) can be one of the compounds disclosed in U.S. Patent Application No. 2004/0127518 (The Regents of the University of California).

In a preferred embodiment, the FAAH inhibitor can be a compound of Formula (IV):

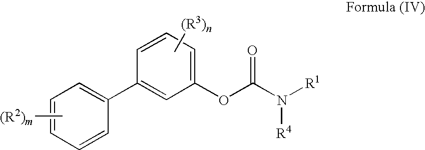

where:
R¹ is selected from among C₁-C₈ alkyl, C₁-C₄ alkyl-(C₃-C₈ cycloalkyl), and C₃-C₈ cycloalkyl (e.g., cyclohexyl);
R⁴ is H or alkyl;
R² and R³ are each independently selected from among H, C₁-C₄ alkyl, C₂-C₄ alkenyl, C₂-C₄ alkynyl, C₃-C₆ cycloalkyl, C₁-C₄alkyl-(C₃-C₆cycloalkyl), aryl, substituted aryl, arylalkyl, —C(O)R^{A}, hydroxy-(C₁-C₆ alkyl), amino-(C₁-C₆ alkyl), —CH₂—NR^{A}R^{B}, —O—(C₁-C₄), arloxy, halo, C₁-C₆-haloalkyl, cyano, hydroxy, nitro, amino, —C(O)NR^{A}R^{B}, ONR^{A}R^{B}, —O—C(O)NR^{A}R^{B}, —SO₂NR^{A}R^{B};
R^{A} and R^{B} are each independently selected from among hydrogen, C₁-C₆ alkyl, and C₃-C₆ cycloalkyl; and m and n are each independently 0-3; or a pharmaceutically acceptable salt thereof.

In a particular embodiment, the FAAH inhibitor has the structure of compound KDS-4103:

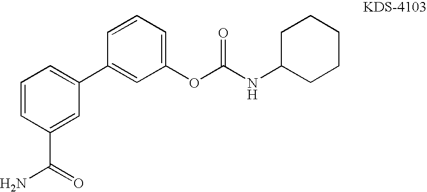

In other embodiments, the FAAH inhibitor is selected from those disclosed in US Patent Application 2007/0155707 (Kadmus Pharmaceuticals, Inc.) and in US Patent Application 2007/0155747 (Kadmus Pharmaceuticals, Inc.).

Suitable FAAH inhibitor compositions have also been described in U.S. Pat. Nos. 6,462,054 (The Scripps Research Institute) and 6,891,043 (The Scripps Research Institute), US patent application US20070004741 (Johnson & Johnson), and in the International Patent Applications WO04020430 (Sanofi-Synthelabo), WO04067498 (Sanofi-Synthelabo), WO04099176 (Sanofi-Synthelabo), WO05033066 (Sanofi-Aventis), WO2006117461 (Sanofi-Aventis), WO02087569 (Bristol-Myers Squibb Company), WO03065989 (Bristol-Myers Squibb Company), WO9749667 (The Scripps Research Institute), WO9926584 (The Scripps Research Institute), WO04033652 (The Scripps Research Institute), WO06044617 (The Scripps Research Institute), WO07098142 (The Scripps Research Institute), WO2006054652 (Takeda), WO2006074025 (Janssen Pharma), WO2007061862 (Janssen Pharma), WO2006088075 (Astellas Pharma) and in the Japanese patent application JP2006306746 (Astellas Pharma).

The composition containing the FAAH inhibitor can be administered orally.

In addition to being administered a FAAH inhibitor, the subject can also be administered a therapeutically effective amount of oleolylethanolamide, palmitoylethanolamide, or a combination thereof.

In some embodiments, the subject suffers from a depressive disorder or an anxiety disorder.

In one embodiment, a triglyceride-restricted diet is provided to the subject. In addition, the subject's triglyceride levels can be monitored.

In further embodiments, the subject can be provided a calorie-restricted diet or an exercise regimen.

Unless defined otherwise, all technical and scientific terms used herein have the same meaning as is commonly understood by one of skill in the art to which the claimed subject matter belongs.

The term “acceptable” with respect to a formulation, composition or ingredient, as used herein, means having no persistent detrimental effect on the general health of the subject being treated.

As used herein, the term “agonist” refers to a compound, the presence of which results in a biological activity of a protein that is the same as the biological activity resulting from the presence of a naturally occurring ligand for the protein. As used herein, the term “partial agonist” refers to a compound the presence of which results in a biological activity of a protein that is of the same type as that resulting from the presence of a naturally occurring ligand for the protein, but of a lower magnitude.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, may be branched, straight chain, or cyclic. Depending on the structure, an alkyl group can be a monoradical or a diradical (i.e., an alkylene group).

As used herein, C₁-Cₓ includes C₁-C₂, C₁-C₃ . . . C₁-Cₓ.

The “alkyl” moiety may have 1 to 10 carbon atoms (whenever it appears herein, a numerical range such as “1 to 10” refers to each integer in the given range; e.g., “1 to 10 carbon atoms” means that the alkyl group may have 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups can be substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which may be the same or different. Non-limiting examples of an alkynyl group include —C≡CH, —C≡CCH₃ and —C≡CCH₂CH₃. The “R” portion of the alkynyl moiety may be branched, straight chain, or cyclic. Depending on the structure, an alkynyl group can be a monoradical or a diradical (i.e., an alkynylene group). Alkynyl groups can be optionally substituted.

As used herein, the term “antagonist” refers to a compound, the presence of which results in a decrease in the magnitude of a biological activity of a protein. In certain embodiments, the presence of an antagonist results in complete inhibition of a biological activity of a protein, such as, for example, fatty acid amide hydrolase. In certain embodiments, an antagonist is an inhibitor.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

An “aryloxy” group refers to an (aryl)O— group, where aryl is as defined herein. The terms “co-administration” or the like, as used herein, are meant to encompass administration of the selected therapeutic agents to a single patient, and are intended to include treatment regimens in which the agents are administered by the same or different route of administration or at the same or different time.

As used herein, the term “cyano” refers to a group of formula —CN.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Depending on the structure, a cycloalkyl group can be a monoradical or a diradical (e.g., an cycloalkylene group). As used herein, the term “carbocycle” refers to a ring, wherein each of the atoms forming the ring is a carbon atom. Carbocylic rings can be formed by three, four, five, six, seven, eight, nine, or more than nine carbon atoms. Carbocycles can be optionally substituted.

As used herein, “EC50” refers to a dosage, concentration or amount of a particular test compound that elicits a dose-dependent response at 50% of maximal expression of a particular response that is induced, provoked or potentiated by the particular test compound.

The term “effective amount,” refers to the amount of an active FAAH inhibitor composition that is required to confer a therapeutic or cosmetic effect on the subject. A “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “halo” or, alternatively, “halogen” or “halide” means fluoro, chloro, bromo or iodo.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

As used herein, the “IC₅₀” refers to an amount, concentration or dosage of a particular test compound that achieves a 50% inhibition of a maximal response, such as inhibition of FAAH, in an assay that measures such response.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, mercapto, alkylthio, arylthio, alkylsulfoxide, arylsulfoxide, alkylsulfone, arylsulfone, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutically acceptable salt” refers to a formulation of a compound that does not cause significant irritation to an organism to which it is administered and does not abrogate the biological activity and properties of the compound. Pharmaceutically acceptable salts may be obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts also may be obtained by reacting a compound described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art.

A “subject,” as referred to herein, can be any vertebrate (e.g., a mouse, rat, cat, guinea pig, hamster, rabbit, zebrafish, dog, non-human primate, or human) unless specified otherwise.

Other features, objects, and advantages will be apparent from the description and from the claims.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph showing plasma lipoprotein fractionation profiles from animals in a control group and orally administered oleoylethanolamide (500 mg/kg) and KDS-4103 groups.

FIG. 2 is a representative plot depicting in cynomolgus monkeys the time course of the mean OEA plasma concentration following different oral doses of KDS 4103.

### DETAILED DESCRIPTION

There is an ongoing need for compositions and methods that can control the risk factors that underlie EMDs. Accordingly, methods are described herein for treating a subject suffering from an EMD. Methods for reducing body fat, caloric intake, and body weight are also described.

Unless defined otherwise, all technical and scientific terms used herein have the same meaning as is commonly understood by one of skill in the art to which the claimed subject matter belongs.

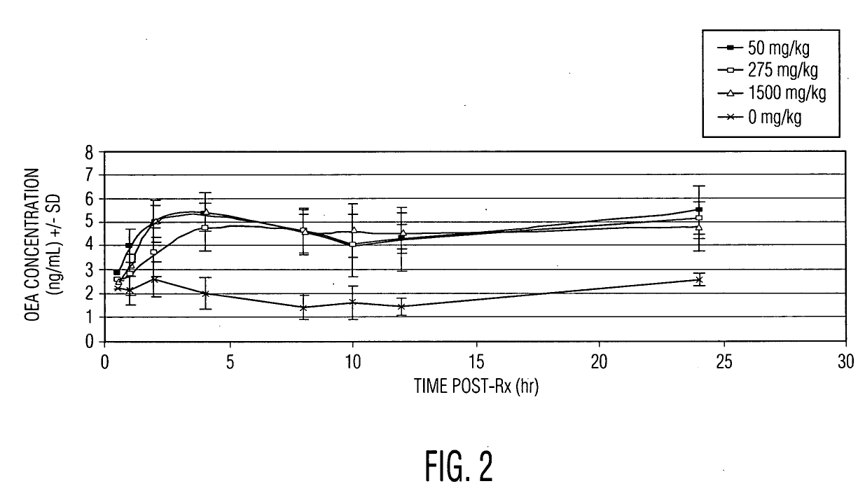

The methods described herein include administering a composition containing an amount of a FAAH inhibitor sufficient to increase systemic levels of one or more fatty acid amides, e.g., oleoylethanolamide (OEA), palmitoylethanolamide (PEA), or anandamide (AEA) to therapeutically or cosmetically effective levels. See, e.g., FIG. 2. Without being bound by theory, it is thought that certain fatty acid amides, such as OEA act through the peroxisome proliferator-activated receptor a (PPAR-α) to regulate diverse physiological processes, including, e.g., feeding and lipolysis. Consistent with this, human adipose tissue has been shown to bind and metabolize endocannabinoids such as anandamide and 2-arachidonylglycerol. See Spoto et al., Aug. 22, 2006, Biochimie (E-publication ahead of print); and Matias et al. (2006), J. Clin. Endocrin. & Met., 91(8):3171-3180. Thus, inhibiting FAAH activity in vivo leads to reduced body fat, body weight, caloric intake, and liver triglyceride levels. However, unlike, other anti-lipidemic agents that act through PPAR-α, e.g., fibrates, FAAH inhibitors do not cause adverse side effects such as rash, fatigue, headache, erectile dysfunction, and, more rarely, anemia, leukopenia, angioedema, and hepatitis. See, e.g., Muscari et al (2002), Cardiology, 97:115-121. Anadditional therapeutic property of FAAH inhibitors, is that due to their ability to elevate anandamide levels, they effectively alleviate depression and anxiety, conditions often associated with EMDs such as obesity. See Simon et al. (2006), Archives of Gen. Psychiatry, 63(7):824-830. Finally, agonism of cannabinoid receptors has also been shown to reduce the progression of atherosclerosis in animal models. See Steffens et al. (2005), Nature, 434:782-786; and Steffens et al. (2006), Curr. Opin. Lipid., 17:519-526. Thus, increasing the level of endogenous cannabinergic fatty acid amides (e.g., anandamide) is expected to effectively treat or reduce the risk of developing atherosclerosis.

Accordingly, as described herein, FAAH inhibitors can used to reduce to treat or reduce the risk of EMDs, which include, but are not limited to, obesity, appetite disorders, overweight, cellulite, Type I and Type II diabetes, hyperglycemia, dyslipidemia, steatohepatitis, liver steatosis, non-alcoholic steatohepatitis, Syndrome X, insulin resistance, diabetic dyslipidemia, anorexia, bulimia, anorexia nervosa, hyperlipidemia, hypertriglyceridemia, atherosclerosis, arteriosclerosis, inflammatory disorders or conditions, Alzheimers disease, Crohn's disease, vascular inflammation, inflammatory bowel disorders, rheumatoid arthritis, asthma, thrombosis, or cachexia

The methods described herein can be used to treat, e.g., insulin resistance syndrome and diabetes, i.e., both primary essential diabetes such as Type I Diabetes or Type II Diabetes and secondary nonessential diabetes. Administering a composition containing a therapeutically effective amount of an in vivo FAAH inhibitor reduces the severity of a symptom of diabetes or the risk of developing a symptom of diabetes, such as atherosclerosis, hypertension, hyperlipidemia, liver steatosis, nephropathy, neuropathy, retinopathy, foot ulceration, or cataracts.

In another embodiment, the methods described herein are used to treat food abuse behaviors, especially those liable to cause excess weight, e.g., bulimia, appetite for sugars or fats, and non-insulin-dependent diabetes.

In some embodiments, the subject to be treated, in addition to suffering from an EMD, also suffers from a depressive disorder or from an anxiety disorder. Preferably, the subject is diagnosed as suffering from the depressive or psychiatric disorder prior to administration of the FAAH inhibitor composition. Thus, a dose of a FAAH inhibitor that is therapeutically effective for both the EMD and the depressive or anxiety disorder is administered to the subject. Methods for treatment of anxiety and depressive disorders by FAAH inhibition are described in, e.g., U.S. Patent Application Nos. 2004/0127518 (The Regents of the University of California), 2007/0155707 (Kadmus Pharmaceuticals, Inc.) and 2007/0155747 (Kadmus Pharmaceuticals, Inc.).

Preferably, the subject to be treated is human. However, the methods can also be used to treat non-human mammals. Animal models of EMDs such as those described in, e.g., U.S. Pat. No. 6,946,491 (Wellstat Therapeutics Corporation) are particularly useful.

FAAH inhibitors can also be used for the manufacture of a medicament for treating any of the foregoing conditions.

Symptoms, diagnostic tests, and prognostic tests for each of the above-mentioned conditions are known in the art. See, e.g., “Harrison's Principles of Internal Medicine©,” 16th ed., 2004, The McGraw-Hill Companies, Inc., and the “Diagnostic and Statistical Manual of Mental Disorders©,” 4th ed., 1994, American Psychiatric Association.

FAAH inhibitor compositions can also be used to decrease body-weight in individuals wishing to decrease their body weight for cosmetic, but not necessarily medical considerations.

A FAAH inhibitor composition can be administered in combination with a drug for lowering circulating cholesterol levels (e.g., statins, niacin, fibric acid derivatives, or bile acid binding resins). FAAH inhibitor compositions can also be used in combination with a therapeutically or cosmetically effective amount of a weight loss drug, e.g., an appetite suppressant such as diethylpropion, mazindole phendimetrazine, phentermine, or sibutramine; hyperlipidemia drugs (such as statins and PPAR agonists); obesity drugs; fibric acids lipid up-take blockers (eg. Orlistat); MCH-1 (Melanin-concentrating hormone) antagonists, or nesfatin-1 agonists.

The methods described herein can also include providing an exercise regimen or providing a calorie-restricted diet (e.g., a triglyceride-restricted diet) to the subject.

Candidate in vivo FAAH inhibitors can be identified by their ability to increase systemic levels of one or more FAAs. Suitable FAAs include fatty acid ethanolamides with a fatty acid moiety containing 14 to 28 carbons, with 0 to 6 double bonds, such as OEA, PEA, AEA, and stearoylethanolamide (SEA). Other suitable FAAs include primary fatty acid amides with a fatty acid moiety containing 14 to 28 carbons, with 0 to 6 double bonds, such as oleamide. Biological samples from which FAA levels can be assayed are, e.g., plasma, serum, blood, cerebrospinal fluid, saliva, or urine.

FAA levels in a biological sample are assayed, e.g., by liquid chromatography tandem-mass spectrometry (LC-MS/MS). Increased assay reproducibility is achieved by spiking biological samples with a known amount of an isotopically labeled FAA, which serves as an internal standard for the FAA to be assayed. The level of the FAA can also be determined using spectrophotometric techniques (e.g., a fluorometric method). Alternatively, the level of the FAA can be determined using a biological assay. In some embodiments, the level of the FAA is determined using a combination of the aforementioned techniques. Any of the foregoing assays for FAA levels can be partly or fully automated for high throughput. Details of this and other FAA assays, as well as methods for analyzing changes in FAA levels are known in the art. See, e.g., Quistad et al. (2002), Toxicology and Applied Pharmacology 179: 57-63; Quistad et al. (2001), Toxicology and Applied Pharmacology 173, 48-55; Boger et al. (2000), Proc. Natl. Acad. Sci. U.S.A. 97, 5044-49; Cravatt et al. Proc. Natl. Acad. Sci. U.S.A. 98, 9371-9376 (2001); Ramarao et al. (2005), Anal. Biochem. 343: 143-51.

### Examples of FAAH Inhibitors

The FAAH inhibitor compositions used in the methods described herein can come from a variety of sources including both natural (e.g., plant extracts) and synthetic.

A FAAH inhibitor used in the methods described herein can inhibit FAAH activity, in vitro, with an IC₅₀ of less than 10 μM (e.g., 1 μM, 0.5 μM, or 0.01 μM).

### Methods of Dosing and Treatment Regimens

The compounds described herein can be used in the preparation of medicaments for the inhibition of fatty acid amide hydrolase, or for the treatment of diseases or conditions that would benefit, at least in part, from inhibition of fatty acid amide hydrolase. In addition, a method for treating any of the diseases or conditions described herein in a subject in need of such treatment, involves administration of pharmaceutical compositions containing at least one FAAH inhibitor or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

The amount of a given agent that will correspond to such an amount will vary depending upon factors such as the particular compound, disease or condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. For example, the starting level of one or more FAAs can vary between individuals, and within individuals, e.g., according to a fasting state or disease state. In general, however, doses employed for adult human treatment will typically be in the range of 0.02-5000 mg per day, preferably 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

### Combination Treatments

The compositions and methods described herein may also be used in conjunction with other well known therapeutic reagents that are selected for their particular usefulness against the condition that is being treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the disease, disorder, or condition, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

In addition, the compounds described herein also may be used in combination with procedures that may provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a mutant gene that is known to be correlated with certain diseases or conditions.

The compounds described herein and combination therapies can be administered before, during or after the occurrence of a disease or condition, and the timing of administering the composition containing a compound can vary. Thus, for example, the compounds can be used as a prophylactic and can be administered continuously to subjects with a propensity to develop conditions or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, preferably within the first 48 hours of the onset of the symptoms, more preferably within the first 6 hours of the onset of the symptoms, and most preferably within 3 hours of the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. A compound is preferably administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria. For example, the compound or a formulation containing the compound can be administered for at least 2 weeks, preferably about 1 month to about 5 years, and more preferably from about 1 month to about 3 years.

### EXAMPLES

The following specific examples are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet Reference thereto evidences the availability and public dissemination of such information.

### Example 1

### FAAH Inhibitors Reduce Body Weight Body Fat, and Liver Steatosis

We assessed the effect of inhibiting FAAH activity on body weight, body fat, triglyceride levels, cholesterol levels in APOE*3-Leiden transgenic (E3L) mice, an animal model of hyperlipidemia. E3L mice express a mutated variant of human apoE, apoE*3-Leiden, that has impaired binding of apoE to the LDL receptor. Consequently, E3L mice exhibit a decreased clearance rate of apoB-containing lipoproteins and elevated serum lipid levels. See van Vlijmen et al. (1994), J. Clin Invest., 93:1403-1410. Indeed, upon high fat and cholesterol feeding, these mice develop various stages of atherosclerotic lesions depending on plasma total cholesterol levels and resembling those found in humans. See Groot et al. (1996), Arterioscler. Thromb. Vasc. Biol., 16:926-933; Versch ren et al. (2005), Arterioscler. Thromb. Vasc Biol., 25:161-167; and Lutgens et al. (1999), Circulation; 99(2):276-283). Thus, the E3L mouse is a suitable model for the investigation of the efficacy of anti-atherosclerotic drugs. Accordingly, we evaluated the effects of a FAAH inhibitor (KDS 4103) in E3L mice.

E3L mice were fed a high cholesterol (1% w/w) diet (HC diet) for a period of four weeks. Animals were then matched based on their plasma cholesterol levels, and were divided into five groups, each of which was maintained on an HC diet. Every day for the remainder of the study (four weeks), a “control” group received food with no additives, a “fenofibrate” group received food containing fenofibrate (0.04% w/w), an “oral vehicle” group received an oral suspension of vehicle, an “oral OEA” group received an oral suspension of OEA at a dose of 500 mg/kg, and an “oral KDS 4103” group received an oral suspension of KDS-4103 at a dose of 10 mg/kg. The composition of the vehicle and KDS-4103 suspensions are shown in Table 1.

TABLE 1
Composition of KDS-4103 and Vehicle Suspensions

| | OEA | | KDS-4103 | | Vehicle | |
|---|---|---|---|---|---|---|
| COMPONENT | Suspension | | Suspension | | Suspension | |
| OEA | 750 | mg | — | | — | |
| KDS-4103 | — | | 15 | mg | — | |
| sodium carboxymethyl | 50 | mg | 50 | mg | 50 | mg |
| cellulose | | | | | | |
| Tween-80 | 40 | mg | 40 | mg | 40 | mg |
| water | 9.160 | g | 9.895 | g | 9.910 | g |

Blood samples were collected at days 0, 14, and 28 of the treatment period. At the end of the treatment period, animals were sacrificed, and various tissues and organs were analyzed. Unless otherwise indicated, values are mean±standard deviation (SD).

TABLE 2
Organ and Tissue Weights (g)

| Organ | Control | Fenofibrate | Oral Vehicle | Oral OEA | Oral KDS-4103 |
|---|---|---|---|---|---|
| Brain | 0.43 ± 0.04 | 0.46 ± 0.04 | 0.44 ± 0.01 | 0.44 ± 0.02 | ^{#}0.46 ± 0.02 |
| Fat | 0.47 ± 0.13 | 0.48 ± 0.14 | 0.35 ± 0.09 | *0.24 ± 0.06 | *0.22 ± 0.04 |
| Heart | 0.12 ± 0.01 | 0.14 ± 0.02 | 0.13 ± 0.02 | 0.14 ± 0.01 | 0.14 ± 0.02 |
| Liver | 1.44 ± 0.14 | ^{#}1.77 ± 0.28 | 1.01 ± 0.15 | 0.91 ± 0.16 | 1.03 ± 0.14 |
| Lung | 0.21 ± 0.03 | 0.21 ± 0.05 | 0.18 ± 0.02 | 0.19 ± 0.02 | 0.18 ± 0.01 |

^{#}Statistically significant increase (p < 0.05) compared to control group
*Statistically significant decrease (p < 0.05) compared to control group

As shown in Table 2, gonadal fat weight was significantly lower in the KDS-4103 group and OEA groups relative to the “oral vehicle” group, indicating that KDS-4103 and OEA reduced body fat stores. A slight, but significant increase was observed in brain weight in the KDS-4103 group. No significant changes were observed in heart, liver, or lung weights in the KDS-4103 group. Fenofibrate treatment resulted in a significant increase in liver weight, but had no effect on brain, fat, heart or lung weights relative to its control group.

Body weights were determined at multiple time points, and, as shown in table 3, were found to be significantly reduced within the KDS-4103 and OEA groups at the last time point examined (4 weeks).

TABLE 3
Body Weight (g)

| | | | Oral | Oral | Oral |
|---|---|---|---|---|---|
| Week | Control | FF | Vehicle | OEA | KDS-4103 |
| 0 | 21.9 ± 1.2 | 23.3 ± 1.6 | 21.9 ± 0.9 | 22.3 ± 1.3 | 21.8 ± 1.1 |
| 0.5 | ^{#}22.3 ± 1.1 | 23.3 ± 2.0 | 22.1 ± 1.0 | 22.5 ± 1.0 | 21.9 ± 1.0 |
| 1 | ^{#}22.5 ± 1.1 | 23.7 ± 2.0 | 21.9 ± 1.0 | 22.6 ± 1.0 | 21.9 ± 1.0 |
| 1.5 | ^{#}22.5 ± 1.2 | 23.6 ± 2.0 | 21.4 ± 0.7 | 21.8 ± 1.1 | *21.3 ± 0.9 |
| 2 | 22.2 ± 1.1 | 23.4 ± 2.1 | 21.7 ± 1.6 | 21.9 ± 1.2 | *21.4 ± 0.9 |
| 2.5 | 22.4 ± 1.2 | 23.5 ± 1.9 | 21.7 ± 0.8 | 21.9 ± 1.3 | *21.4 ± 0.9 |
| 3 | 22.3 ± 1.0 | 23.4 ± 1.8 | 21.6 ± 1.0 | *21.6 ± 1.1 | *21.3 ± 0.9 |
| 3.5 | 22.5 ± 1.1 | 23.7 ± 1.8 | 21.4 ± 1.1 | *21.3 ± 1.2 | 21.5 ± 0.9 |
| 4 | ^{#}22.6 ± 1.2 | 23.5 ± 1.7 | 21.4 ± 1.0 | *21.4 ± 1.2 | *21.5 ± 1.0 |

^{#}Statistically significant increase (p < 0.05) compared to t = 0 weeks within group
*Statistically significant decrease (p < 0.05) compared to t = 0 weeks within group

Food intake per animal per day (at the cage level) was determined at 0-4 weeks, as shown in Table 4. A slight, but significant, decrease in food intake was observed for both the OEA and KDS-4103 groups at the last time point.

TABLE 4
Food Intake Per Animal Per Day (g)

| Interval | | Oral | Oral |
|---|---|---|---|
| (weeks) | Control | OEA | KDS-4103 |
| run-in | 2.5 ± 0.5 | 2.5 ± 0.5 | 2.5 ± 0.5 |
| 0-0.5 | 2.5 ± 0.1 | 2.5 ± 0.05 | 2.3 ± 0.1 |
| 0.5-1.0 | 2.3 ± 0.1 | 2.3 ± 0.04 | 2.4 ± 0.1 |
| 1.0-1.5 | 2.3 ± 0.03 | 2.4 ± 0.1 | 2.3 ± 0.2 |
| 1.5-2.0 | 2.5 ± 0.4 | 2.4 ± 0.1 | 2.6 ± 0.1 |
| 2.0-2.5 | 2.0 ± 0.03 | 2.3 ± 0.1 | 2.3 ± 0.3 |
| 2.5-3.0 | 2.2 ± 0.1 | 2.3 ± 0.05 | 2.5 ± 0.1 |
| 3.0-3.5 | 2.2 ± 0.2 | 2.3 ± 0.1 | 2.4 ± 0.3 |
| 3.5-4.0 | 2.4 ± 0.1 | *2.0 ± 0.1 | *2.0 ± 0.1 |

^{#}Statistically significant decrease (p < 0.05) compared to t = 0 weeks within group

Total plasma cholesterol levels were determined on days 0, 14, and 28, as shown in Table 5.

TABLE 5
Total Plasma Cholesterol (mM)

| | | | Oral | Oral | Oral |
|---|---|---|---|---|---|
| Day | Control | Fenofibrate | Vehicle | OEA | KDS-4103 |
| 0 | 13.2 ± 3.3 | 13.6 ± 4.2 | 13.8 ± 1.6 | 13.9 ± 3.4 | 14.3 ± 3.2 |
| 14 | 15.8 ± 1.9 | *6.5 ± 1.0 | 13.2 ± 2.6 | ^{#}16.0 ± 2.9 | ^{#}19.1 ± 4.9 |
| 28 | 15.3 ± 2.6 | *6.2 ± 1.3 | 13.6 ± 1.8 | ^{#}15.8 ± 2.7 | ^{#}19.5 ± 4.1 |

^{#}Statistically significant increase (p < 0.05) compared to t = 0 within group and compared to respective group
*Statistically significant decrease (p < 0.05) compared to t = 0 within group and compared to respective control group

Total plasma cholesterol levels on days 14 and 28 were slightly and significantly increased in the KDS-4103 and OEA groups relative to the oral vehicle group. Total plasma cholesterol levels on days 14 and 28 were significantly decreased in the fenofibrate group relative to its control group.

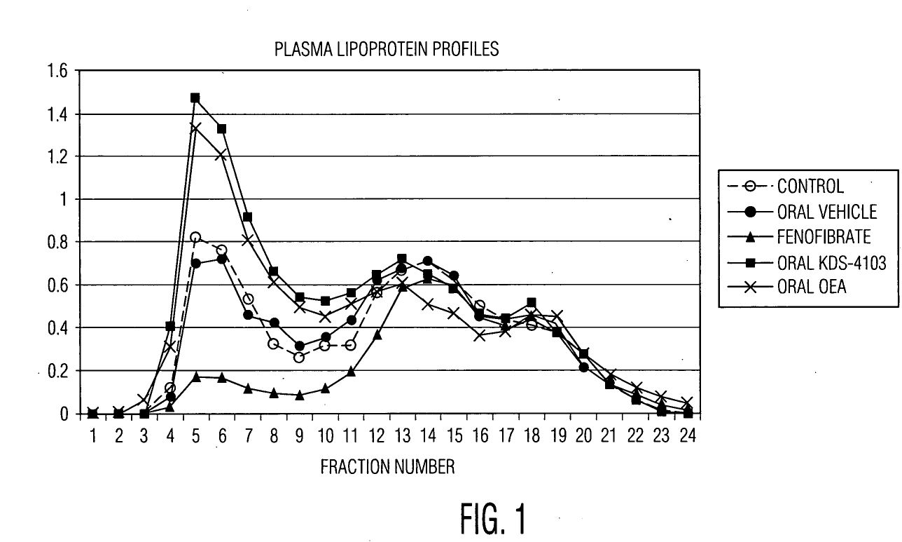

Lipoprotein profiles were determined in pooled plasma from day 28, as described in Verschuren et al. (2005), Arterioscler. Thromb. Vasc Biol., 25:161-167. An equal amount of plasma per animal was used to prepare a plasma pool for each group. Plasma lipoproteins in each plasma pool were separated by size fractionation. Cholesterol concentrations of the fractions are shown in FIG. 1. Typically, VLDL, IDL/LDL and HDL lipoproteins are eluted in fractions 4-8, fractions 11-16 and fractions 17-20, respectively.

Treatment with KDS-4103 or OEA resulted in an increase of the VLDL fraction. Treatment with KDS-4103 or OEA was also associated with a slight shift toward larger IDL/LDL particles possibly indicating that KDS-4103 may have slightly suppressed the formation of LDL particles from VLDL. Treatment with fenofibrate resulted in decreases in the VLDL and LDL fractions.

Total plasma triglyceride levels were determined on days 0, 14, and 28, as shown in table 6.

TABLE 6
Total Plasma Triglyceride Levels (mM)

| | | | Oral | Oral | Oral |
|---|---|---|---|---|---|
| Day | Control | Fenofibrate | Vehicle | OEA | KDS-4103 |
| 0 | 1.7 ± 0.7 | 1.6 ± 0.4 | 1.7 ± 0.4 | 1.5 ± 0.4 | 1.5 ± 0.4 |
| 14 | 1.7 ± 0.2 | ^{#}*0.7 ± 0.2 | 2.0 ± 0.7 | 1.7 ± 0.6 | 1.9 ± 0.6 |
| 28 | 1.7 ± 0.4 | ^{#}*0.7 ± 0.2 | 1.6 ± 0.3 | *1.3 ± 0.3 | 1.9 ± 0.5 |

^{#}Statistically significant decrease (p < 0.05) compared to t = 0 within group
*Statistically significant decrease (p < 0.05) compared to respective control group

Total plasma triglyceride levels were slightly decreased in the oral OEA group relative to the oral vehicle group, but were not significantly changed in the Oral KDS-4103 group relative to the oral vehicle group. Fenofibrate treatment caused a substantial, and significant decrease in total plasma triglyceride levels when compared to its control group and the within group measurement obtained at the first time point.

Levels of plasma alanine aminotransferase (ALAT) were determined on day 28 (table 7). Plasma ALAT is a measure of hepatic stress and increases in plasma ALAT levels are often associated with liver damage. Indeed, animals fed a HC diet have roughly three times the level of plasma ALAT of animals fed a normal chow diet. Thus, a reduction in plasma ALAT levels is associated with improved liver function.

TABLE 7
Plasma Alanine Amino Transferase Activity (U/L) at Day 28

| | | | Oral | Oral | Oral |
|---|---|---|---|---|---|
| Day | Control | Fenofibrate | Vehicle | OEA | KDS-4103 |
| 28 | 135.9 ± | 112.5 ± 10.4 | 174.5 ± 18.3 | *68.6 ± 17.6 | *94.0 ± 22.1 |
| | 14.8 | | | | |

*Statistically significant decrease (p < 0.05) compared to respective control group

Plasma ALAT levels were significantly decreased in the KDS-4103 group when compared to the control vehicle group. No significant change in plasma ALAT was observed in the fenofibrate group when compared to its control group.

In order to examine cellular stress in the liver due to lipid storage (steatosis), we stained frozen liver tissue sections with Oil Red O a fat-soluble diazo dye used to stain for lipids. Digital photomicrographs of liver cross sections were taken for each individual animal (magnification: 20 fold). Photomicrographs were analyzed blindly using morphometric software (QWin, Leica) as follows: the intensity of the Oil Red O-staining (red color) was measured as a percentage of a randomly selected hepatic area (vascular structures do not stain with Oil Red O and were also not selected) and above a threshold of 175 relative units and 145 relative units. In this semi-quantitative analysis, the intensity above 175 relative units was termed ‘severe steatotic area’ and the intensity above 145 relative units was termed ‘severe and mild steatotic area.

TABLE 8
Oil Red O Staining in Liver
(% of cross-sectional area)

| Staining | | | |
|---|---|---|---|
| Intensity | Oral Vehicle | Oral OEA | Oral KDS-4103 |
| Mild-Severe | 54 ± 5 | *36 ± 5 | 53 ± 4 |
| (above 115 | | | |
| relative units) | | | |
| Severe | 14 ± 2 | *8 ± 2 | 10 ± 1 |
| (above 175 | | | |
| relative units) | | | |

*Statistically significant decrease (p < 0.05) compared to control group

As shown in table 8, treatment with OEA resulted in a small, but significant reduction in steatotic staining, when compared to the control vehicle group. KDS-4103 treatment resulted in a small, but not significant decrease (p=0.055) in staining particularly in the percentage of severe steatotic staining area.

A reduction of Red Oil O staining in liver generally indicates reduced liver lipid content. We therefore quantified levels of lipids in livers from each animal, as described in Havekes et al. (1987), Biochem. J., 247:739-746; Delsing et al. (2005), J. Cardiovasc. Pharmacol., 45:53-60; and Post et al. (2004), ATVB, 24:768-774. Liver tissue homogenates were prepared. Lipids were extracted, dried under nitrogen, dissolved in chloroform, and spotted onto a channeled 20×20 cm silica gel thin layer chromatography (TLC) plate. Lipids were then separated, TLC plates were warmed up to visualize bands, and bands were quantified using an internal standard as reference. The results are shown in Table 9.

TABLE 9
Lipid Content of Liver

| | | | Oral | Oral | Oral |
|---|---|---|---|---|---|
| Lipid | Control | Fenofibrate | Vehicle | OEA | KDS-4103 |
| Free | 22 ± 2 | 21 ± 2 | 22 ± 2 | 21 ± 2 | 22 ± 2 |
| Cholesterol | | | | | |
| Cholesterol | 46 ± 5 | 44 ± 6 | 48 ± 5 | ^{#}61 ± 7 | ^{#}59 ± 6 |
| Esters | | | | | |
| Triglycerides | 77 ± 7 | *107 ± 6 | 98 ± 6 | 94 ± 11 | *64 ± 14 |

^{#}Statistically significant increase (p < 0.05) compared to respective control group
*Statistically significant decrease (p < 0.05) compared to respective control group

Levels of free cholesterol (FC) were not significantly changed in any of the treatment groups. Levels of cholesterol esters (CE) were significantly increased in the KDS-4103 and the OEA group when compared to the oral vehicle group. Levels of triglycerides (TG) were significantly decreased in the KDS-4103 group when compared to the control vehicle group and were significantly increased in the fenofibrate group when compared to its control group.

Free cholesterol (FC) is an essential component of cellular membranes, but increases in FC can generally be toxic, particularly in tissues such as liver. The formation of CE is a mechanism for processing FC, and increases in the esterification of surplus cholesterol into the CE pool can serve as a protective response. Disruption of the synthesis, transport and removal of long-chain fatty acids and TG are the basis for the development of liver steatosis, including non-alcoholic fatty liver disease (NAFLD). Steatosis occurs when the rate of import or synthesis of fatty acids exceeds the rate of export or catabolism. As a result, increases in TG levels may indicate the development of steatosis and decreases in TG levels may indicate an opposite effect.

The effects of KDS-4103 and OEA treatment on the lipid content of the E3L mouse livers (no change in FC, increase in CE, decrease in TG) were consistent with a decrease in steatosis and an improvement of liver health.

Leptin is a peptide hormone that is a key regulator of body weight. Recent studies with obese and non-obese humans demonstrated a strong positive correlation of serum leptin concentrations with percentage of body fat. It appears that as adipocytes increase in size due to accumulation of triglyceride, they synthesize more and more leptin. In essence, leptin provides the body with an index of nutritional status.

Accordingly, we quantified plasma leptin levels after a four hour fasting period at treatment time points 0, 14, and 28 days using a mouse-specific leptin ELISA or (kit number MOB00 from R&D Systems, Minneapolis, Minn.) according to the guidelines and protocol specified by the manufacturer. As shown in table 10, oral KDS-4103 significantly decreased plasma leptin levels relative to those in the oral vehicle control group. In the OEA group, plasma leptin levels were lower at 14 days relative to the oral vehicle control group. The effect of KDS-4103 on plasma leptin levels is consistent with its effect on body fat (as shown in table 2).

TABLE 10
Plasma Leptin Levels (ng/ml)

| | Oral | Oral | Oral |
|---|---|---|---|
| Day | Vehicle | OEA | KDS-4103 |
| 0 | 6.2 ± 1.1 | 4.7 ± 2.3 | ^{#}3.9 ± 1.5 |
| 14 | 11.1 ± 2.8 | ^{#}7.3 ± 3.1 | ^{#}4.5 ± 2.1 |
| 28 | 7.2 ± 1.6 | 6.1 ± 4.6 | ^{#}5.1 ± 2.3 |

^{#}Statistically significant decrease (p < 0.05) compared to control group

Adiponectin is a hormone produced exclusively by adipocytes. Adiponectin production and its plasma serum concentrations are decreased in a variety of obese and insulin-resistant states. On the other hand, adiponectin has anti-atherogenic properties among which the capacity to inhibit monocyte adhesion to endothelial cells (see reviewed by M. Guerre-Millo (2004), Diabetes, 30:13-19; N. Mendez-Sanchez (2006), Mini Rev Med. Chem. 2006 6:651-656.

We determined plasma adiponectin levels after a four hour fasting period at 0, 14, 28 days, using a mouse-specific adiponectin/Acrp30 ELISA (kit number MRP300 from R&D Systems, Minneapolis, Minn.). As shown in table 11, plasma adiponectin concentrations remained more or less constant, except for the oral KDS-4103 group, which had a significantly higher level of plasma adiponectin at day 28 as compared to day 0.

TABLE 11
Plasma Adiponectin Levels (mg/ml)

| | Oral | Oral | Oral |
|---|---|---|---|
| Day | Vehicle | OEA | KDS-4103 |
| 0 | 11.8 ± 2.6 | 12.9 ± 2.8 | 10.4 ± 1.9 |
| 14 | 12.7 ± 3.5 | 11.3 ± 3.5 | 10.2 ± 2.7 |
| 28 | 12.2 ± 1.9 | 11.4 ± 2.0 | ^{#}11.9 ± 1.9 |

^{#}Statistically significant increase (p < 0.05) compared to t = 0 within group

Ketone bodies (acetoacetate, β-hydroxybutyrate (HB) and acetone) can become major body fuels during fasting and consumption of ketogenic diets (high fat, low carbohydrate). HB is the main metabolic product in ketoacidosis and is a better measurement of the degree of ketosis than serum ketones (Trachtenbarg (2005), Am Fam Physician., 71:1705-1714). The circulating levels of ketone bodies are determined by their rates of production (ketogenesis) and utilization (ketolysis). During fasting, most ketone bodies arise from long-chain fatty acids liberated from adipose tissue (lipolysis). Lipolysis is extremely sensitive to suppression by insulin. In some of the groups of this study, high insulin levels were paralleled by low levels of ketone bodies.

We quantified plasma D-3-hydroxybutyrate (b-HBA; ketone bodies) levels were quantified in plasma samples obtained after a 4-hour fasting period at time points t=0, t=2, and t=4 weeks using kits #2940 (b-HBA reagent set) and #2947 (b-HBA control set)) according to the guidelines and protocol specified by the manufacturer (Instruchemie, Delfzijl, The Netherlands). As shown in table 12, treatment with OEA or KDS-4103 resulted at the end of the treatment period (at 28 days) in significantly lower HB levels when compared to the oral vehicle control group.

TABLE 12
Plasma Ketone Body (HB) levels (mM)

| | Oral | Oral | Oral |
|---|---|---|---|
| Day | Vehicle | OEA | KDS-4103 |
| 0 | 0.60 ± 0.18 | 0.79 ± 0.19 | 0.73 ± 0.24 |
| 14 | 0.20 ± 0.11 | 0.23 ± 0.09 | 0.32 ± 0.11 |
| 28 | 0.67 ± 0.20 | ^{#}0.38 ± 0.10 | ^{#}0.43 ± 0.11 |

^{#}Statistically significant decrease (p < 0.05) compared to control group

Based on these studies, we concluded that inhibiting FAAH activity (e.g., by administering KDS-4103 or other FAAH inhibitors described herein), or otherwise increasing the level of FAAs (such as, e.g., administering OEA), decreased body fat, body weight, and reduced liver steatosis. Further, we conclude that inhibition of FAAH activity (a) altered fatty acid metabolism, (b) altered or increased lipolysis, (c) altered plasma cholesterol levels, (d) altered cholesterol metabolism, (e) reduced plasma ALAT, and (f) reduced plasma markers of liver toxicity.

### Example 2

### KDS-4103 Causes Prolonged Elevation of Plasma OEA Levels in Primates

We wished to examine the ability of KDS-4103 to increase OEA levels in primates. KDS-4103 at doses of 50-1500 mg/kg, or vehicle, was administered orally to cynomolgus monkeys. Afterwards, a time course of plasma OEA levels was determined for each subject by obtaining a plasma sample at 0.5, 1, 2, 4, 8, 12, and 24 hours post-administration. As shown in FIG. 2, in subjects administered each dose of KDS-4103, plasma OEA levels were clearly elevated two hours after administration, peaked at four hours, and remained elevated at all subsequent time points examined. On the basis of these data, we concluded that KDS-4103 is highly effective for increasing OEA levels in primates.

### Example 3

### Long-Term Effects in E3L Mice on a High Fat Diet

Analysis of the long-term effects of a FAAH inhibitor is performed in an animal model that is predictive of the human disease process, the E3L mouse model.

Male E3L mice are treated with a diabetogenic high fat diet for 4-6 weeks. These mice are then divided into several different treatment groups. Mice are then treated with no treatment (food control), vehicle or KDS-4103. KDS-4103 is administered at doses of 10 mg/kg, 30 mg/kg, 50 mg/kg, or 100 mg/kg. Treatments continue for 16 weeks.

KDS-4103 is formulated as a suspension as in Example 1. Separately, KDS-4103 is formulated in the diet in amounts (w/w) that result in 10, 30, 50, or 100 mg/kg doses per day.

Body weight (individually) and food intake (per cage) are determined throughout the study. Plasma is collected for lipid analyses at weeks 0, 4, 8, 12, and 16. The following parameters are determined in plasma samples: total cholesterol, total triglycerides, free fatty acids, leptin, adiponectin, lipoprotein distribution and ALAT. At sacrifice, tissue samples (including liver, muscle, and adipose) are collected, weighed, and frozen. Oral glucose tolerance tests are performed at weeks 0, 8, and 16.

### Example 4

### Long-Term Effects in E3L Mice on a High Cholesterol Diet

Analysis of the long-term effects of a FAAH inhibitor is performed in an animal model that is predictive of the human disease process, the E3L mouse model. E3L mice have intact lipoprotein metabolism, and female E3L mice develop signs of hyperlipidemia and atherosclerosis when feeding on a high cholesterol diet.

Male E3L mice are treated with a cholesterol-containing atherogenic diet for 4-6 weeks. These mice are then divided into several different treatment groups. Mice are then treated with no treatment (food control), vehicle or KDS-4103. KDS-4103 is administered at doses of 10 mg/kg, 30 mg/kg, 50 mg/kg, or 100 mg/kg. Treatments continue for 16 weeks.

KDS-4103 is formulated as a suspension as in Example 1. Separately KDS-4103 is formulated in the diet as in Example 2.

Assessments: body weight (individually) and food intake (on cage level) are determined throughout the study. Plasma is collected for lipid analyses at week 0, 4, 8, 12 and 16. The following parameters are determined in plasma samples: total cholesterol, total triglycerides, free fatty acids, E-selectin, lipoprotein distribution and ALAT. At sacrifice, tissue samples (including aorta, liver, muscle and adipose) are collected, weighed, and frozen. Hearts and aortas are evaluated morphologically and morphometrically to determine the extent and degree (severity) of atherosclerosis, using 4 cross-sections per animal. Cross sections are prepared from paraffin-embedded aortic root areas with standardized intervals of 50 μm).

A number of embodiments of the invention have been described. Nevertheless, it will be understood that various modifications may be made without departing from the spirit and scope of the invention. Accordingly, other embodiments are within the scope of the following claims.

1. A method for treating an energy metabolism disorder in a subject in need thereof, the method comprising administering to the subject a composition containing a therapeutically effective amount of an inhibitor of fatty acid amide hydrolase activity.

2. A method for reducing body fat in a subject in need thereof, the method comprising administering to the subject a composition containing a therapeutically effective amount of an inhibitor of fatty acid amide hydrolase activity.

3. A method for reducing body weight of a subject in need thereof, the method comprising administering to the subject a composition containing a cosmetically effective amount of an inhibitor of fatty acid amide hydrolase activity.

4. A method for reducing caloric intake in a subject in need thereof, the method comprising administering to the subject a composition containing a therapeutically effective amount of an inhibitor of fatty acid amide hydrolase activity.

5. The method of claim 1, wherein the inhibitor is a carbamate derivative fatty acid amide hydrolase inhibitor.

6. The method of claim 5, wherein the inhibitor is a compound of Formula IV): 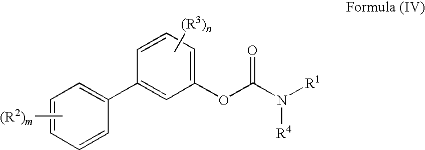
where:
R¹ is selected from among C₁-C₈ alkyl, C₁-C₄ alkyl-(C₃-C₈ cycloalkyl), and C₃-C₈ cycloalkyl (e.g., cyclohexyl); R⁴ is H or alkyl;
R² and R³ are each independently selected from among H, C₁-C₄ alkyl, C₂-C₄ alkenyl, C₂-C₄ alkynyl, C₃-C₆ cycloalkyl, C₁-C₄ alkyl-(C₃-C₆ cycloalkyl), aryl, substituted aryl, arylalkyl, —C(O)R^{A}, hydroxy-(C₁-C₆ alkyl), amino-(C₁-C₆ alkyl), —CH₂—NR^{A}R^{B}, —O—(C₁-C₄), arloxy, halo, C₁-C₆-haloalkyl, cyano, hydroxy, nitro, amino, —C(O)NR^{A}R^{B}, —ONR^{A}R^{B}, —O—C(O)NR^{A}R^{B}, —SO₂NR^{A}R^{B};
R^{A} and R^{B} are each independently selected from among hydrogen, C₁-C₆ alkyl, and C₃-C₆ cycloalkyl; and m and n are each independently 0-3; or a pharmaceutically acceptable salt thereof.

7. The method of claim 6, wherein R¹ is cyclohexyl.

8. The method of claim 6, wherein the compound of Formula (IV) has the structure of compound KDS-4103:

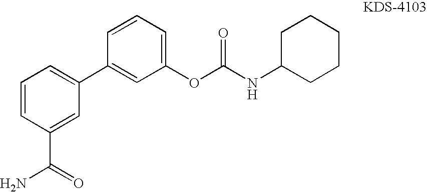

9. The method of claim 1, wherein the energy metabolism disorder is insulin resistance, diabetes, hyperlipidemia, liver steatosis, steatohepatitis, non-alcoholic steatohepatitis, arteriosclerosis, or atherosclerosis.

10. The method of claim 9, wherein the energy metabolism disorder is insulin resistance.

11. The method of claim 9, wherein the energy metabolism disorder is diabetes.

12. The method of claim 9, wherein the energy metabolism disorder is hyperlipidemia.

13. The method of claim 9, wherein the energy metabolism disorder is obesity.

14. The method of claim 9, wherein the energy metabolism disorder is arteriosclerosis.

15. The method of claim 9, wherein the energy metabolism disorder is liver steatosis, steatohepatitis, or non-alcoholic steatohepatitis.

16. The method of claim 12, further comprising administering a therapeutically effective amount of a drug for lowering circulating cholesterol levels to the subject.

17. The method of claim 16, wherein the drug for lowering cholesterol levels is a statin, niacin, fibric acid derivative, or bile acid binding resin.

18. The method of claim 17, wherein the drug for lowering cholesterol levels is a statin.
